(19) United States
(12) Patent Application Publication
(10) Pub. No.: US 2012/0058232 A1
(43) Pub. Date: Mar. 8, 2012

(54) Method for the preparation of diacetyl tartaric acid esters of mono-and di-glycerides

(76) Inventors: Gaofeng Liu, Guangdong (CN); Shuming Yu, Guangdong (CN)

(21) Appl. No.: 13/143,933

(22) PCT Filed: Sep. 27, 2010
(86) PCT No.: PCT/CN10/77359

§ 371 Date: Sep. 19, 2011

(30) Foreign Application Priority Data
Feb. 10, 2010 (CN) 201010112359.X

(51) Int. Cl. A21D 2/16; C07C 67/08

(52) U.S. Cl. 426/330; 554/164

(57) ABSTRACT

The present invention provides a method for the preparation of diacetyl tartaric acid esters of mono- and di-glycerides, which comprises steps of: adding L-tartaric acid and/or DL-tartaric acid and acetic anhydride into a reaction kettle, adding concentrated phosphoric acid as catalyst and mixing together to form a reaction system; heating the reaction kettle until a temperature of the reaction system reaches 50° C.˜55° C. to initiate a reaction, after the temperature of the reaction system rises and then drops to 80° C.˜90° C., keeping the temperature at 80° C.˜90° C. for 20˜60 min to obtain an intermediate product; adding stearic acid esters of mono- and di-glycerides to the intermediate product, then adding powdery sodium hydroxide as catalyst, controlling an inner pressure of the reaction kettle at −0.09 Mpa˜−0.098 Mpa, heating the reaction kettle again until a temperature of the reaction system reaches 90° C.˜130° C., and keeping the temperature at 90° C.˜130° C. for 20˜40 min to obtain said diacetyl tartaric acid esters of mono- and di-glycerides. The preparation method according to the present invention has advantages of simple process, low production cost, high yield, good product quality and environmental friendliness.

### TECHNICAL FIELD

The present invention relates to the field of food additives, and especially relates to a new method for the preparation of a food emulsifier.

### BACKGROUND OF THE INVENTION

Food emulsifiers are a kind of food additive, which are used in food manufacture to mix immiscible liquids (e.g. oil and water) to form a stable emulsion. Such emulsifiers could be absorbed and excreted by human body, they have no adverse effects on metabolism and would not accumulate in the body to influence human health. Food emulsifiers usually have no peculiar sapor and have no influence on the sapor of the food productions, so they can be widely used in the manufacture of different kinds of food. There are varieties of food emulsifiers, which approximately account for half amount of food additives. Food emulsifiers are a kind of additive that are used most in food industry. In recent years, diacetyl tartaric acid esters of mono- and di-glycerides (DATEM), as the third generation of internationally accepted food emulsifiers, are used in baked food in foreign countries. After DATEM is added in wheat flour food, the hydrophilic groups of DATEM would combine with the gliadin of the wheat gluten and the hydrophobic groups of DATEM would combine with the gluten of the wheat gluten, thus a compound of gluten protein is formed to make gluten network closer and more elastic, so that the gas retention of the fermented dough, the fermentation endurance and the rapid swelling ability could be enhanced, also the volume of the baked products could be expanded. Diacetyl tartaric acid esters of mono- and di-glycerides (DATEM) are not only emulsifiers, but also good stabilizers. However, high requirement for the equipments used in DATEM manufacture results in a high price of the products.

A thesis named “Preparation of Food Emulsifier DATEM” (Journal of Wuxi University of Light Industry, 1996, (4): 308-312) has disclosed that ring-opening reaction of acetic anhydride and its esterification reaction with tartaric acid could be initiated by acetic acid, to form a mixture of diacetyl tartaric acid and diacetyl tartaric acid anhydride, and such mixture could react with fatty glyceride to produce DATEM. However, this DATEM preparation method has several defects. First, the catalytic activity of acetic acid is quite low, after the reaction temperature reached 80˜90° C. by external heating, the system temperature would rise rapidly in a short time as the reaction progresses, which would easily impact the kettle and affect safety in production. Second, a low reaction efficiency results in a mixture of diacetyl tartaric acid and diacetyl tartaric acid anhydride, and the final product quality would fall off because it is difficult for diacetyl tartaric acid to react with stearic acid monoglyceride. Third, the production cost is too high for industrial application. Fourth, a flash heat release at high temperature during the reaction would lead to a black appearance for the products and even produce black resin by-products, but no diacetyl tartaric acid anhydride.

Chinese patent CN99815233.1 has disclosed a mixture of diacetyl tartaric acid esters of mono- and di-glycerides based on C12 to C22 fatty acids. The product could be obtained by slowly adding food grade L-(+)tartaric acid to acetic anhydride with trace amounts of concentrated sulphuric acid as catalyst; reacting in oil bath and then removing the residual acetic anhydride and acetic acid through reduced pressure distillation; mixing the melt fatty acid monoglyceride with 4 wt % diglycerides and diacetyl tartaric acid anhydride at a high speed of 24000 rmp under dry nitrogen protection for reaction; then adding sodium stearate as catalyst, dispersing at a high speed for 20 minutes and cooling on a metal plate; and finally powdered by cryogenic milling. It can be proved by experiments that in the presence of concentrated sulphuric acid, the reaction between tartaric acid and acetic anhydride is an exothermic reaction, and tartaric acid is easy to be decomposed and condensed. When tartaric acid is added and heated by oil bath to a temperature of 140˜150° C., the vacuum degree must be controlled under −0.08 Mpa, otherwise no diacetyl tartaric acid anhydride but black or brunette liquids are produced in the condition of strong acid, high temperature and air, even though in the presence of inert gas. Moreover, high speed mixing and reacting brings more difficulty to process operation, and nitrogen protection would increase production costs which goes against industrial application.

From the above, it can be known that the existing DATEM production technologies have several technical problems: high production costs caused by high requirement for reaction conditions which would go against industrial application; low product yield, poor quality and no DATEM obtained under the industrial production conditions; significant environmental pollution during the production processes.

### SUMMARY OF THE INVENTION

To solve at least one said technical problems, the present invention provides a new method for the preparation of diacetyl tartaric acid esters of mono- and di-glycerides (DATEM) which has simple process, low production cost, high yield, good product quality and environmental friendliness.

A method for the preparation of diacetyl tartaric acid esters of mono- and di-glycerides according to the present invention comprises steps of:
A. adding L-tartaric acid and/or DL-tartaric acid and acetic anhydride into a reaction kettle, adding concentrated phosphoric acid as catalyst and mixing together to form a reaction system;
B. heating the reaction kettle until a temperature of the reaction system reaches 50° C.˜55° C. to initiate a reaction, after the temperature of the reaction system rises and then drops to 80° C.˜90° C., keeping the temperature at 80° C.˜90° C. for 20˜60 min to obtain an intermediate product; and
C. adding stearic acid esters of mono- and di-glycerides to the intermediate product, then adding powdery sodium hydroxide as catalyst, controlling an inner pressure of the reaction kettle at −0.09 Mpa˜−0.098 Mpa, heating the reaction kettle again until a temperature of the reaction system reaches 90° C.˜130° C., and keeping the temperature at 90° C.˜130° C. for 20˜40 min to obtain said diacetyl tartaric acid esters of mono- and di-glycerides.

The present invention uses esterification reaction in the presence of concentrated phosphoric acid as catalyst to make diacetyl tartaric acid esters of mono- and di-glycerides. Tartaric acid has three types of conformation: D-tartaric acid, L-tartaric acid and DL-tartaric acid. D-tartaric acid can not be used as food additive because of its toxicity, thus the present invention uses L-tartaric acid, DL-tartaric acid or a mixture thereof according to certain ratio, and especially uses DL-tartaric acid as one reactant. Another reactant is acetic anhydride. The following reaction will occur between the two reactants under the catalysis of concentrated phosphoric acid at the temperature of 50° C.˜55° C.

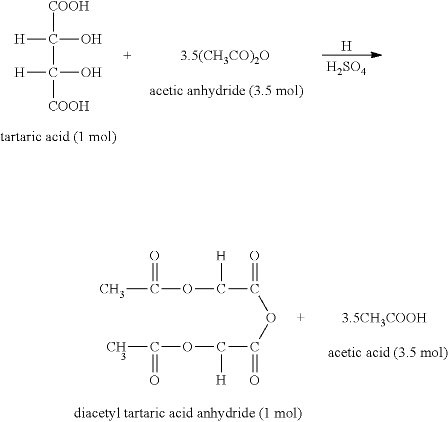

The yield and purity of diacetyl tartaric acid anhydride obtained by the above reaction would directly affect the quality, yield and application effect of the final products. In the present invention, the yield of diacetyl tartaric acid anhydride approximates 98%, and the melting point ranges from 120° C. to 132° C., preferably from 128° C. to 130° C., which means that the present invention has the advantages of high purity and high yield, and no diacetyl tartaric acid is produced, which is difficult to esterify with glycerol stearate.

In the prior art, concentrated sulphuric acid is used as catalyst in the reaction, but such reaction is too intense to control, it would easily impact the kettle, cause risks for the safety in production, and lead to a black appearance and poor quality for the products. In the present invention, concentrated phosphoric acid is used as catalyst in step A, which brings advantages of milder reaction, less side reaction, easier control and no influence on product appearance and quality. The phosphoric acid used in the present invention has a concentration of more than 85%. In one embodiment, the amount of concentrated phosphoric acid accounts for 0.004%˜0.005% of the total amount of L-tartaric acid and/or DL-tartaric acid. In the step B of the present invention, the reactants begin to react with each other when the temperature of the reaction system rises to a certain degree (e.g. 50° C.˜55° C.) by external heating. Ring-opening reaction of acetic anhydride progresses automatically. Heat is released as the reaction progresses and the temperature of the reaction system continues to rise under the reaction heat. Usually, the highest temperature of the reaction system can reach 120° C., but the temperature would fall down after that. When the temperature drops to 80° C.˜90° C., keep the temperature for a certain while, usually 20˜60 min, to improve completeness of the reaction. In one embodiment, the temperature of 80° C.˜90° C. is kept for 30˜40 min.

According to the preparation method of the present invention, a weight ratio between acetic anhydride and L-tartaric acid and/or DL-tartaric acid is 1.5˜3.5:1. The actual amount of acetic anhydride is higher than its theoretical amount, so as to increase the reaction yield.

According to one embodiment of the present invention, acetic anhydride is added into the reaction system at least in twice. For example, acetic anhydride could be added into the reaction system in twice, in three times or more according to the total amount calculated from said ratio. Acetic anhydride is added by portions to avoid rapid rising of the reaction temperature which would impact the kettle.

The intermediate product formed in step B mainly contains diacetyl tartaric acid anhydride and a by-product acetic acid. It is different from the prior art that diacetyl tartaric acid anhydride and the by-product acetic acid do not need to be separated, thus the operations of acetic acid distillation or crystallization, centrifugation, washing, re-centrifugation, drying and storing in dark at low temperature can be avoided, so that the investment for equipments and the production costs could be reduced, and the production environment could be improved. Another reactant used in the present invention is stearic acid esters of mono- and di-glycerides which have a freezing point of 60° C.˜70° C. and an iodine value not more than 3 g/100 g, wherein the amount of stearic acid monoglyceride accounts for 50%˜90% of the total amount of stearic acid esters of mono- and di-glycerides. According to one embodiment of the present invention, the amount of stearic acid monoglyceride accounts for 70%˜80% of the total amount of stearic acid esters of mono- and di-glycerides.

In step C, stearic acid esters of mono- and di-glycerides are added to the intermediate product that contains diacetyl tartaric acid anhydride, then powdery sodium hydroxide is added as catalyst. According to one embodiment of the present invention, the amount of powdery sodium hydroxide accounts for 0.004%˜0.005% of the total amount of L-tartaric acid and/or DL-tartaric acid.

When the inner pressure of the reaction kettle is reduced to vacuum, the reaction kettle is heated for a second time until the temperature of the esterification reaction system reaches 90° C.˜130° C.. The equation of the main esterification reaction is as follows.

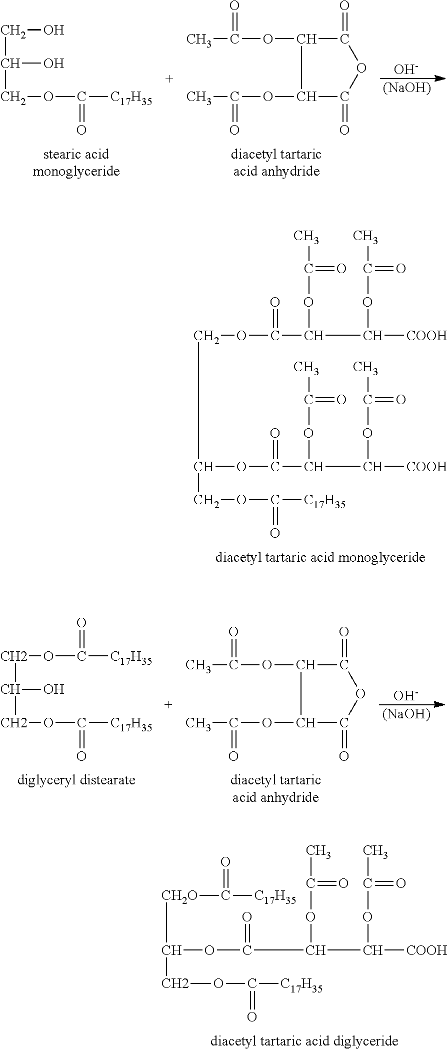

The by-product acetic acid and some water are removed as the esterification reaction progresses. The acetic acid and water in the reaction kettle can be removed completely in the condition that the inner pressure of the reaction kettle is controlled at −0.09 Mpa˜−0.098 Mpa and the distillation temperature reaches 125° C.

According to one embodiment of the present invention, a weight ratio between stearic acid esters of mono- and di-glycerides added in step C and L-tartaric acid and/or DL-tartaric acid added in step A is 2.5˜4:1. The amount of powdery sodium hydroxide accounts for 0.004%˜0.005% of the total amount of L-tartaric acid and/or DL-tartaric acid.

The content of sodium hydroxide within the powdery sodium hydroxide is above 90%. The powdery sodium hydroxide is used as catalyst to avoid extra moisture brought into the reaction system.

The diacetyl tartaric acid esters of mono- and di-glycerides produced by the preparation method of the present invention have an acid value of 60˜105 mgKOH/g and a saponification value of 300˜550 mgKOH/g. The acid value and saponification value of the products can be monitored to reflect the progress degree of the reaction, and the reaction would be accomplished when the above values are achieved.

According to one embodiment of the present invention, after the reaction is accomplished, the diacetyl tartaric acid esters of mono- and di-glycerides are prilled by spray at the temperature of 90° C.˜110° C. under the atmospheric pressure in step C, to make a white solid form thereof. Further, the present invention provides an application of the diacetyl tartaric acid esters of mono- and di-glycerides added with anticaking agents as baked food emulsifiers. A series of baked food emulsifiers with different quality and good fluency can be obtained by mixing the DATEM solid and anticaking agents.

According to one embodiment of the present invention, said anticaking agent includes but is not limited to at least one of the 90% stearic acid monoglyceride, fluent starch and food grade silicon dioxide and tricalcium phosphate.

The method for the preparation of diacetyl tartaric acid esters of mono- and di-glycerides according to the present invention has following advantages.

(1) Advantages of simple processes, mild reaction conditions, strong controllability and high safety are achieved.
(2) Equipment invest is low because it has no need for high temperature and high pressure reaction kettle, and no need for rapid mixing of melted diacetyl tartaric acid anhydride and fatty acid esters of mono- and di-glycerides.
(3) In the prior art, inert gas must be used to prevent the products from oxidation which would lead to a black appearance for the product. However in the present invention, the acetic acid gas produced from the reaction is enough to prevent the air from entering the reaction system, which could reduce the production costs because it has no need for inert gas protection.
(4) Environmental pollution is low because a leaking of the by-product acetic acid gas during the crystallization and centrifugation processes can be avoided.

The production cost for the diacetyl tartaric acid esters of mono- and di-glycerides (DATEM) produced by the preparation method of the present invention has been greatly reduced, equal to a half of the production cost for the same products by the foreign countries. The products provided by the present invention have good fluency, fine appearance and high quality, which accord with the international standard for the product quality.

Further aspects and advantages of the present invention would be described below, which will make them clearer and easier to understand.

### BRIEF DESCRIPTION OF THE DRAWINGS

Foresaid and/or further aspects and advantages of the present invention would be described in following embodiments accompany with the drawings, to make them clearer and easier to understand.

FIG. 1 is a flow chart of the preparation method according to the present invention.

FIG. 2 is a diagrammatic view of the equipments used for the method according to the present invention.

FIG. 3 is an infra-red spectrogram of the intermediate product diacetyl tartaric acid anhydride formed by the method according to the present invention.

FIG. 4 is an ¹³C-NMR spectrogram of the intermediate product diacetyl tartaric acid anhydride formed by the method according to the present invention.

FIG. 5 is an ¹H-NMR spectrogram of the intermediate product diacetyl tartaric acid anhydride formed by the method according to the present invention.

FIG. 6 is an ¹H-NMR spectrogram of the diacetyl tartaric acid esters of mono- and di-glycerides produced by the method according to the present invention.

### Wherein:

1 represents a measuring tank;
2 represents a mixer;
3 represents a jacket;
4 represents a reaction kettle;
5 represents a freeze-drying granulation device;
6 represents a vacuumizing device; and
7 represents a condenser.

### DETAILED DESCRIPTION OF THE PREFERRED EMBODIMENTS

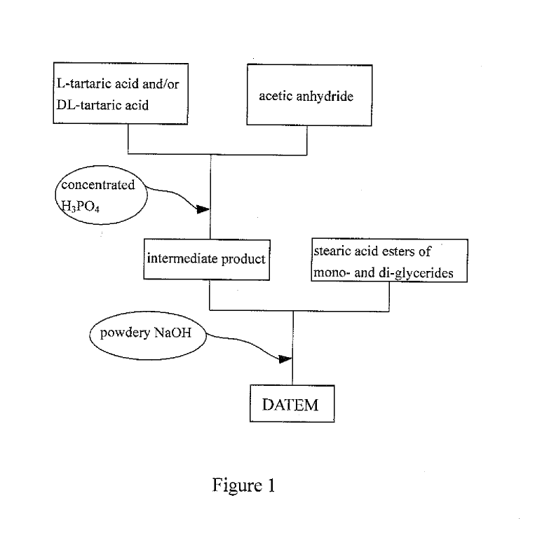

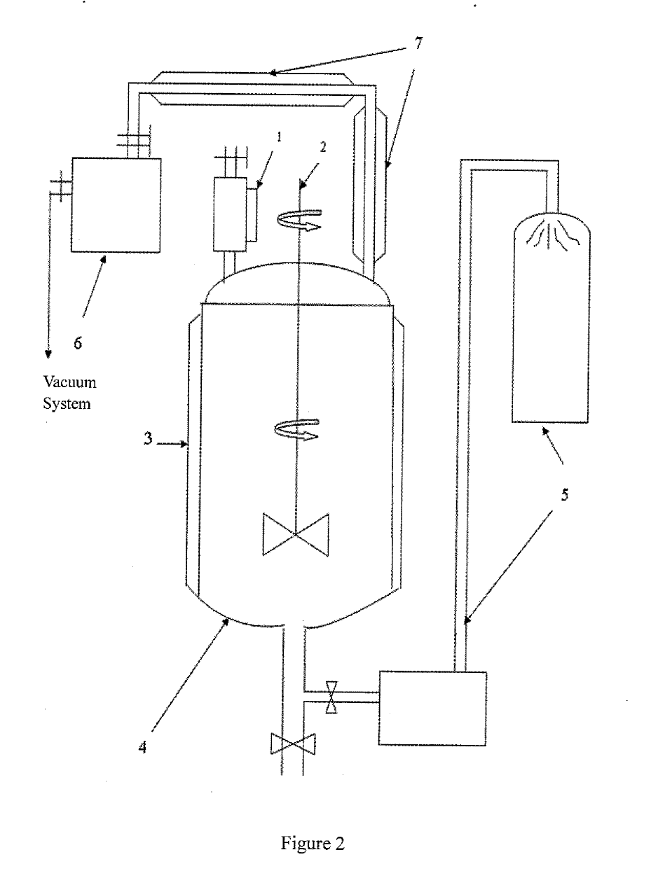

Embodiments of the present invention will be described in details accompany with the drawings. The same reference signs represent the same components or components with the same functions. Embodiments are used to explain the present invention, but not to limit it. The flow of the preparation method according to the present invention will be described accompany with FIG. 1 and FIG. 2. As shown in the figures, a reaction kettle 4 is connected with a measuring tank 1 and a freeze-drying granulation device 5. The reaction kettle 4 is provided with a mixer 2, a jacket 3 and a vacuumizing device 6. During the preparation, the mixer 2 is started to blend the reactants and catalysts, and the jacket 3 is used for heat preservation.

Before the reaction, calculate the amounts of acetic anhydride, concentrated phosphoric acid, stearic acid esters of mono- and di-glycerides and powdery sodium hydroxide according to the amount of L-tartaric acid and/or DL-tartaric acid. Usually, the weight ratio between acetic anhydride and L-tartaric acid and/or DL-tartaric acid is 1.5˜3.5:1, the amount of concentrated phosphoric acid accounts for 0.004%˜0.005% of the total amount of L-tartaric acid and/or DL-tartaric acid, the weight ratio between stearic acid esters of mono- and di-glycerides and L-tartaric acid and/or DL-tartaric acid is 2.5˜4:1, and the amount of powdery sodium hydroxide accounts for 0.004%˜0.005% of the total amount of L-tartaric acid and/or DL-tartaric acid.

Put the reactants into one measuring tank 1, or put the reactants into several measuring tanks 1 respectively. First, put L-tartaric acid and/or DL-tartaric acid and acetic anhydride into the reaction kettle 4, use concentrated phosphoric acid as catalyst, and start the mixer 2 to blend all the materials. According to one embodiment of the present invention, acetic anhydride could be added into the reaction system in several times, for example, in twice or three times, according to the total amount calculated from said ratio, so as to control the reaction process and avoid a rapid rising of the temperature inside the reaction kettle 4.

Start an external heating system. For example, provide the jacket 3 with hot water or provide the jacket 3 with resistance wires for electrical heating. When the inner temperature of the reaction kettle reaches 50° C.˜55° C., the esterification reaction would be initiated and the external heating should be cut off. The esterification reaction is an exothermic reaction, so that the inner temperature of the reaction kettle will rise continually as the reaction progresses. Usually, the highest temperature can reach 120° C., but the temperature would fall down after that. When the temperature drops to 80° C.˜90° C., the temperature could be kept for a certain while under the heat preservation effect of the jacket 3, to improve completeness of the reaction. Usually, the temperature would be kept for 20˜60 min. In one embodiment of the present invention, the temperature is kept for 30˜40 min.

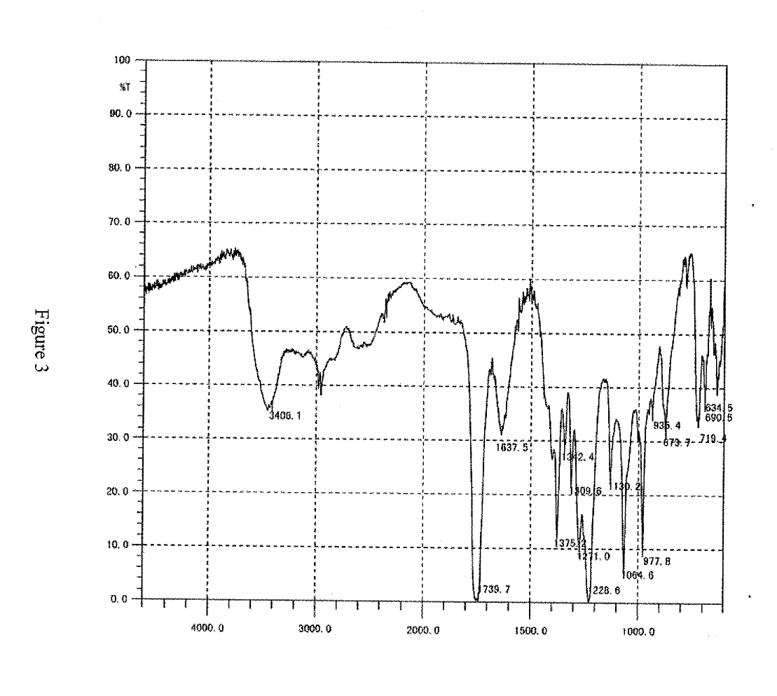

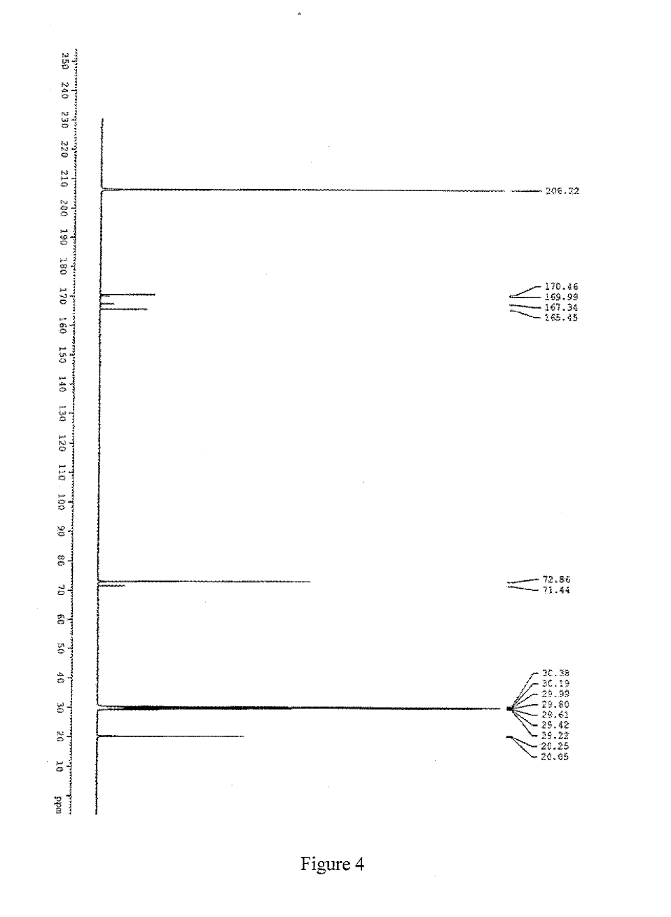

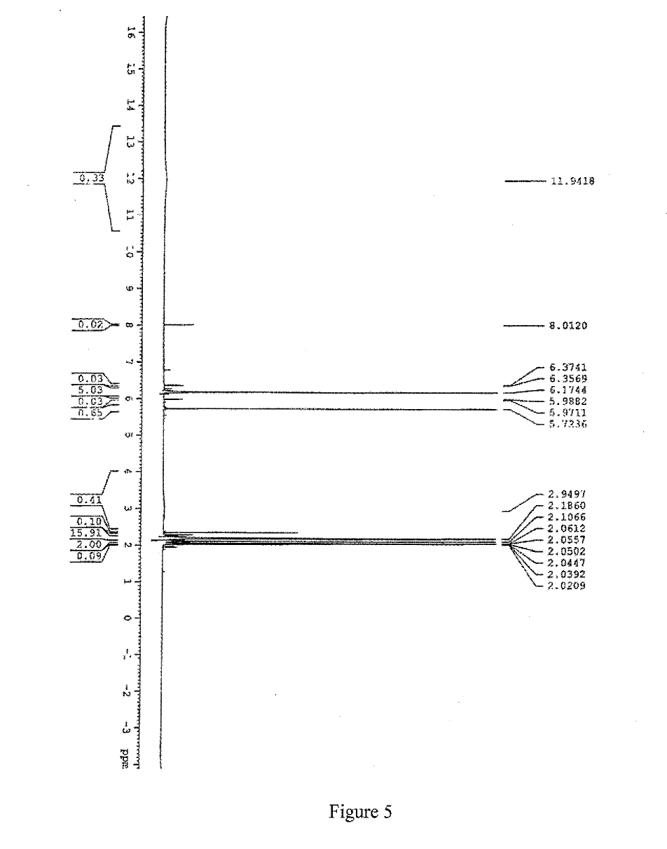

An infra-red spectrogram of the intermediate product produced by the first reaction is shown in FIG. 3. An absorption band with a wavelength of 1739.7 cm⁻¹ (strong) represents a vibration displacement of acetic anhydride which has a formation of five-membered ring. An absorption band with a wavelength of 1228.6 cm⁻¹ (strong) represents a stretching vibration displacement of C═O of acetic ester. It can be confirmed by the infra-red spectrogram that the intermediate product produced by the first reaction is diacetyl tartaric acid anhydride. FIG. 4 is an ¹³C-NMR spectrogram of the intermediate product diacetyl tartaric acid anhydride, and FIG. 5 is an ¹H-NMR spectrogram thereof. FIGS. 3˜5 are characteristic spectrums of diacetyl tartaric acid anhydride. The present invention provides the structure, purity, content and melting point of the intermediate product, because conforming final products can only be obtained by the intermediate product in accordance with the characteristic spectrums.

After the first reaction, put the quantitative stearic acid esters of mono- and di-glycerides into the reaction kettle 4 and add the powdery sodium hydroxide as catalyst. Said stearic acid esters of mono- and di-glycerides are mixtures of stearic acid monoglyceride and stearic acid diglyceride, wherein stearic acid monoglyceride accounts for 50%˜90% of the total amount, preferably 80%, while stearic acid diglyceride accounts for less than 5% thereof. The content of sodium hydroxide within the powdery sodium hydroxide is above 90%, and the powdery sodium hydroxide is used as catalyst to avoid extra moisture brought into the reaction system. After the reactants are added, connect the vacuumizing device 6 with a vacuum system, and heat the reaction kettle again to provide a pressure of −0.09 Mpa˜−0.098 Mpa inside the reaction kettle. When the temperature reaches 90° C.˜130° C., keep the temperature for 20˜40 min. As the temperature rises, the by-product acetic acid and water are removed from the reaction kettle 4 by gasification, and recycled by coagulation in the condenser 7, so as to avoid environmental pollution and improve the operation environment. Usually, no liquid flows out after the temperature reaches 120° C., which means that the by-product acetic acid and water are removed completely.

The primary products of diacetyl tartaric acid esters of mono- and di-glycerides have an appearance of brown oil cream, and the acid value and saponification value thereof are detected to monitor the reaction process. The reaction is basically completed when the acid value of the products reaches 60˜105 mgKOH/g and the saponification value thereof reaches 300˜550 mgKOH/g. The products diacetyl tartaric acid esters of mono- and di-glycerides are mixtures of diacetyl tartaric acid monoglyceride and diacetyl tartaric acid diglyceride, wherein diacetyl tartaric acid diglyceride accounts for less than 4% of the total amount of the products.

Turn off the vacuum system and the vacuumizing device 6, to resume the inner pressure of the reaction kettle to atmospheric pressure. Keep the inner temperature of the reaction kettle 4 at 90° C.˜110° C., start the freeze-drying granulation device 5, and powdery diacetyl tartaric acid esters of mono- and di-glycerides are obtained by spray prilling. The method and equipments disclosed in Chinese Patent 200920001134.X can be used for the spray prilling. Finally, a white solid DATEM is obtained.

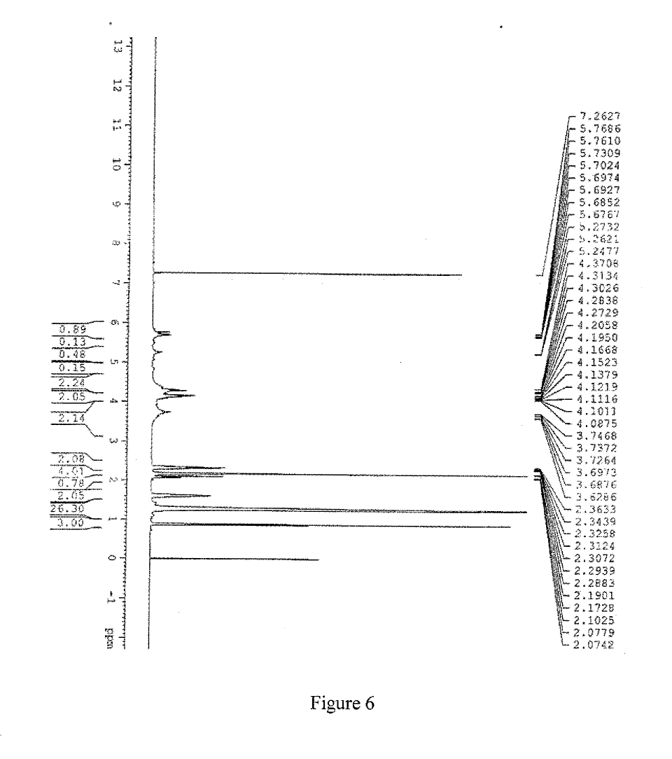

FIG. 6 shows an ¹H-NMR spectrogram of the diacetyl tartaric acid esters of mono- and di-glycerides produced by the present invention. According to the spectrograms, no conforming products are obtained in the situations of similar structure, different components and lower purity. Such spectrograms establish a structure standard for products and semi-products.

A comparison of the acid value and saponification value between the diacetyl tartaric acid esters of mono- and di-glycerides produced by the present invention and other international products with the same chemicals is shown in the following table,

| Name of the product | | Acid value | | Saponification value | |
|---|---|---|---|---|---|
| Danisco | DATEM-205K | 62-76 | mgKOH/g | 380-425 | mgKOH/g |
| Company | DATEM-105K | 42-57 | mgKOH/g | 320-360 | mgKOH/g |
| | DATEM-556K | 62.83 | mgKOH/g | 400.7 | mgKOH/g |
| | DATEM-517K | 72 | mgKOH/g | 440-450 | mgKOH/g |
| | DATEM- | 55.79 | mgKOH/g | 350 | mgKOH/g |
| | A202K | | | | |

| | | | | |
|---|---|---|---|---|
| Kerry Company: | 60.83 | mgKOH/g | 374.61 | mgKOH/g |
| DATEM-868 | | | | |
| Lesaffre Company: | 90-100 | mgKOH/g | 350-450 | mgKOH/g |
| DATEM-322 | | | | |
| European standard | 80-110 | mgKOH/g | 450-510 | mgKOH/g |
| The present invention | 60~105 | mgKOH/g | 300~550 | mgKOH/g |

The products of the present invention are in accordance with the product indexes of the foreign countries, thus in accordance with international market standards.

Basing on foresaid indexes, the diacetyl tartaric acid esters of mono- and di-glycerides produced by the preparation method of the present invention can be used as baked food emulsifiers in the food industry, wherein the diacetyl tartaric acid esters of mono- and di-glycerides are added with anticaking agents. Said anticaking agents include but are not limited to at least one of the 90% stearic acid monoglyceride, fluent starch and food grade silicon dioxide and tricalcium phosphate. A series of food additives with different quality and good fluency can be obtained by mixing the DATEM solid and anticaking agents.

### THE FIRST EMBODIMENT

Acetic anhydride with a content of 99.9% in 100 weight portions, DL-tartaric acid in 125 weight portions and concentrated phosphoric acid with a content of 85% in 0.004 weight portions are added into a reaction kettle under stirring. The jacket is provided with hot water to increase the inner temperature of the kettle to 50° C.˜55° C. An reaction between the materials in the kettle is initiated, and the temperature is increased by the heat released from the reaction. The temperature of the jacket is kept at 55° C. When the inner temperature of the kettle rises to 60° C., the acetic anhydride in 150 weight portions stored in the measuring tank is added into the kettle gradually, and the inner temperature of the kettle would rise to 95° C. ˜120° C. automatically. After all the acetic anhydride stored in a measuring tank has been added, the inner temperature of the kettle drops slowly. The inner temperature of the kettle is kept at 80° C.˜90° C. by heating from the jacket. After 30 min, the reaction would be finished, no suspended solids are left in the kettle and diacetyl tartaric acid anhydride in liquid state is obtained, which is identified as “acid anhydride 1#”.

Stearic acid esters of mono- and di-glycerides with a content of 80% in 464 weight portions and powdery sodium hydroxide in 0.005 weight portions are added into the diacetyl tartaric acid anhydride liquid under stirring. The inner temperature of the kettle drops to 55° C., the kettle is sealed up, the jacket is heated slowly and vacuum distillation is initiated by vacuumizing. When the vacuum degree reaches ˜0.095 Mpa˜−0.098 Mpa, the materials in the kettle start to boil at the temperature of 50° C., and a mass of acetic acid is distilled at the temperature of 90° C. When the distillation speed is slowed down, increase the temperature to 125° C. until no liquid is distilled and keep this state for 30 min. Take samples to inspect. When the acid value of the sample reaches 70±2 mgKOH/g, spray the products at the temperature under 100° C. White powders are obtained by spraying, cooling, drying and prilling, which is identified as “powders 1#”.

### THE SECOND EMBODIMENT

Acetic anhydride with a content of 99.9% in 100 weight portions, DL-tartaric acid in 145 weight portions and concentrated phosphoric acid with a content of 85% in 0.005 weight portions are added into a reaction kettle under stirring. The jacket is provided with hot water to increase the inner temperature of the kettle to 50° C.˜55° C. An reaction between the materials in the kettle is initiated, and the temperature is increased by the heat released from the reaction. The temperature of the jacket is kept at 55° C. When the inner temperature of the kettle rises to 60° C., the acetic anhydride in 190 weight portions stored in the measuring tank is added into the kettle gradually, and the inner temperature of the kettle would rise to 95° C.˜120° C. automatically. After all the acetic anhydride stored in a measuring tank has been added, the inner temperature of the kettle drops slowly. The inner temperature of the kettle is kept at 80° C.˜90° C. by heating from the jacket. After 30 min, the reaction would be finished, no suspended solids are left in the kettle and diacetyl tartaric acid anhydride in liquid state is obtained, which is identified as “acid anhydride 2#”.

Stearic acid esters of mono- and di-glycerides with a content of 80% in 464 weight portions and powdery sodium hydroxide in 0.005 weight portions are added into the diacetyl tartaric acid anhydride liquid under stirring. The inner temperature of the kettle drops to 55° C., the kettle is sealed up, the jacket is heated slowly and vacuum distillation is initiated by vacuumizing. When the vacuum degree reaches ˜0.095 Mpa˜−0.098 Mpa, the materials in the kettle start to boil at the temperature of 55° C., and a mass of acetic acid is distilled at the temperature of 95° C. When the distillation speed is slowed down, increase the temperature to 125° C. until no liquid is distilled and keep this state for 30 min. Take samples to inspect. When the acid value of the sample reaches 72±2 mgKOH/g, spray the products at the temperature under 100° C. White powders are obtained by spraying, cooling, drying and prilling, which is identified as “powders 2#”.

### THE THIRD EMBODIMENT

Acetic anhydride with a content of 99.9% in 170 weight portions, DL-tartaric acid in 180 weight portions and concentrated phosphoric acid with a content of 85% in 0.05 weight portions are added into a reaction kettle under stirring. The jacket is provided with hot water to increase the inner temperature of the kettle to 50° C.˜55° C. An reaction between the materials in the kettle is initiated, and the temperature is increased by the heat released from the reaction. The temperature of the jacket is kept at 55° C. When the inner temperature of the kettle rises to 60° C., the acetic anhydride in 190 weight portions stored in the measuring tank is added into the kettle gradually, and the inner temperature of the kettle would rise to 95° C.˜120° C. automatically. After all the acetic anhydride stored in a measuring tank has been added, the inner temperature of the kettle drops slowly. The inner temperature of the kettle is kept at 80° C.˜90° C. by heating from the jacket. After 30 min, the reaction would be finished, no suspended solids are left in the kettle and diacetyl tartaric acid anhydride in liquid state is obtained, which is identified as “acid anhydride 3#”.

Stearic acid esters of mono- and di-glycerides with a content of 80% in 464 weight portions and powdery sodium hydroxide in 0.005 weight portions are added into the diacetyl tartaric acid anhydride liquid under stirring. The inner temperature of the kettle drops to 50° C., the kettle is sealed up, the jacket is heated slowly and vacuum distillation is initiated by vacuumizing. When the vacuum degree reaches ˜0.095 Mpa˜−0.098 Mpa, the materials in the kettle start to boil at the temperature of 55° C., and a mass of acetic acid is distilled at the temperature of 90° C. When the distillation speed is slowed down, increase the temperature to 125° C. until no liquid is distilled and keep this state for 35 min. Take samples to inspect. When the acid value of the sample reaches 72±2 mgKOH/g, spray the products at the temperature under 100° C. White powders are obtained by spraying, cooling, drying and grilling, which is identified as “powders 3#”.

### THE FOURTH EMBODIMENT

Acetic anhydride with a content of 99.9% in 200 weight portions, DL-tartaric acid in 200 weight portions and concentrated phosphoric acid with a content of 85% in 0.005 weight portions are added into a reaction kettle under stirring. The jacket is provided with hot water to increase the inner temperature of the kettle to 50° C.˜55° C. An reaction between the materials in the kettle is initiated, and the temperature is increased by the heat released from the reaction. The temperature of the jacket is kept at 55° C. When the inner temperature of the kettle rises to 60° C., the acetic anhydride in 200 weight portions stored in the measuring tank is added into the kettle gradually, and the inner temperature of the kettle would rise to 95° C.˜120° C. automatically. After all the acetic anhydride stored in a measuring tank has been added, the inner temperature of the kettle drops slowly. The inner temperature of the kettle is kept at 80° C.˜90° C. by heating from the jacket. After 30 min, the reaction would be finished, no suspended solids are left in the kettle and diacetyl tartaric acid anhydride in liquid state is obtained, which is identified as “acid anhydride 4#”.

Stearic acid esters of mono- and di-glycerides with a content of 80% in 464 weight portions and powdery sodium hydroxide in 0.05 weight portions are added into the diacetyl tartaric acid anhydride liquid under stirring. The inner temperature of the kettle drops to 50° C., the kettle is sealed up, the jacket is heated slowly and vacuum distillation is initiated by vacuumizing. When the vacuum degree reaches −0.095 Mpa˜−0.098 Mpa, the materials in the kettle start to boil at the temperature of 55° C., and a mass of acetic acid is distilled at the temperature of 95° C. When the distillation speed is slowed down, increase the temperature to 125° C. until no liquid is distilled and keep this state for 35 min. Take samples to inspect.

When the acid value of the sample reaches 82±2 mgKOH/g, spray the products at the temperature under 100° C. White powders are obtained by spraying, cooling, drying and prilling, which is identified as “powders 4#”.

### Comparison Test 1

Take 100 g intermediate product diacetyl tartaric acid anhydride from each foresaid embodiment. The intermediate products have a yield of 98% after cooling, crystallization, filtering, washing and vacuum drying. Store the intermediate products in dry brown bottles at 4° C. in dark.

1. Melting point test (by melting point detector): acid anhydride 1#, 2#: m.p120˜123° C.; acid anhydride 3#: m.p128˜130° C.; acid anhydride 4#: m.p129˜132° C.
2. Tartaric acid content (according to FCC method): acid anhydride 1#: 80.0%; acid anhydride 2#: 81.06%; acid anhydride 3#: 83.02%; acid anhydride 4#: 85.12%.
3. Infra-red spectrograms (as shown in FIG. 3).
4. NMR spectrograms (as shown in FIG. 4 and FIG. 5).

### Comparison Test 2

Products can be obtained according to following preparation methods.

A white solid is obtained by spray cooling the powders 1# from the first embodiment, which have an acid value of 65˜70 mgKOH/g, a saponification value of 420˜430 mgKOH/g and a tartaric acid content of 18.46%. DATEM-1# which have an acid value of 64 mgKOH/g, a saponification value of 399 mgKOH/g and a tartaric acid content of 17.5% is obtained by adding 3.5% silicon dioxide and 1.5% tricalcium phosphate as anticaking agents. A white solid is obtained by spray cooling the powders 2# from the second embodiment, which have an acid value of 70.69 mgKOH/g, a saponification value of 456 mgKOH/g and a tartaric acid content of 20.86%. DATEM-2# which have an acid value of 63.90 mgKOH/g, a saponification value of 397.89 mgKOH/g and a tartaric acid content of 18.77% is obtained by adding anticaking agents.

A white solid is obtained by spray cooling the powders 3# from the third embodiment, which have an acid value of 79.87 mgKOH/g, a saponification value of 465.72 mgKOH/g and a tartaric acid content of 24.66%. DATEM-3# which have an acid value of 60.70 mgKOH/g, a saponification value of 384.86 mgKOH/g and a tartaric acid content of 18.74% is obtained by adding anticaking agents.

A white solid is obtained by spray cooling the powders 4# from the fourth embodiment, which have an acid value of 91.10 mgKOH/g, a saponification value of 523.2 mgKOH/g and a tartaric acid content of 26.67%. DATEM-4# which have an acid value of 82.0 mgKOH/g, a saponification value of 470.88 mgKOH/g and a tartaric acid content of 24.00% is obtained by adding anticaking agents.

Said anticaking agents added in powders 1#˜4# contain food grade 90% stearic acid monoglyceride, dry wheat starch and food grade silicon dioxide and tricalcium phosphate.

### Baking Test 1

The differences between the products produced by the present invention and the products produced by the prior art are analysed by a bread baking test. American 556K, American 205K and American Kerry868 are used in controlled groups. Test processes and results are listed as follows.

### Formula:

| Flour | Dry yeast | Sugar | Salt | Grease | Water |
|---|---|---|---|---|---|
| 100 g | 1.2 g | 18 g | 1 g | 6 g | 55 g |

Operation conditions:

| Portion | Intermediate | Final fermentation | | | Baking | | |
|---|---|---|---|---|---|---|---|
| weight | fermentation | Temperature | Humidity | Time | Broiling | Baking | Time |
| 120 g | 15 min | 38° C. | 85% | 115 min | 185° C. | 195° C. | 15 min |

1. Quality evaluation (according to GB/14644-93):

| | | Quality grade | | | | | | | | | |
|---|---|---|---|---|---|---|---|---|---|---|---|
| | | | | | Crust | | | | | | |
| | | Volume | | | character & | | | | | | |
| | | Specific | | Crust | Bread | Crumb | | | | | Total |
| Flour & | | votume | Score | color | traits | color | Smoothness | Texture | Elasticity | Taste | score |
| Additives | | cm³/g | (35) | (5) | (5) | (5) | (10) | (25) | (10) | (5) | (100) |
| 556K | 3000 ppm | 6.20 | 22 | 4 | 3 | 3 | 6 | 18 | 6 | 3 | 65 |
| Strong II A | 300 ppm | | | | | | | | | | |
| 205K | 3000 ppm | 6.76 | 27.6 | 4 | 4 | 3 | 6 | 17 | 8 | 4 | 73.6 |
| Strong II A | 300 ppm | | | | | | | | | | |
| Kerry868 | 4000 ppm | 5.56 | 15.6 | 4 | 3 | 3 | 7 | 18 | 8 | 3 | 61.6 |
| Strong II A | 300 ppm | | | | | | | | | | |
| DATEM-1# | 3000 ppm | 6.42 | 24.2 | 4 | 3 | 4 | 8 | 20 | 8 | 4 | 75.2 |
| Strong II A | 300 ppm | | | | | | | | | | |
| DATEM-2# | 4000 ppm | 6.06 | 20.6 | 4 | 4 | 4 | 8 | 20 | 8 | 4 | 72.6 |
| Strong II A | 300 ppm | | | | | | | | | | |
| DATEM-4# | 3000 ppm | 6.84 | 28.4 | 4 | 4 | 4 | 8 | 20 | 8 | 4 | 80.4 |
| Strong II A | 300 ppm | | | | | | | | | | |

### Analysis:

According to the quality evaluation, the comprehensive quality evaluation results for the baking products produced with different additives are as follows: DATEM-4#>DATEM-1#>205K>DATEM-2#>556K>Kerry868.

2. Toast volume (Kerry868 is not used in the):

| | |
|---|---|
| American 556K: 670/108.0; | American 205K: 730/108.0; |
| DATEM-4#: 740/108.2; | DATEM1#: 700/109.0; |
| DATEM-2#: 730/109.8. | |

3. Bun height (Kerry868 is not used in the):

| | |
|---|---|
| American 556K: 5.46/7.88; | American 205K: 5.42/8.20; |
| DATEM-4#: 5.31/8.83; | DATEM1#: 5.19/8.40; |
| DATEM-2#: 5.42/8.33. | |

4. Texture evaluation:

### DATEM-4#>DATEM1#>DATEM-2#>556K>205K, Kerry868

5. Fermentation time: all are 115 minutes.
6. Others: all the crusts are smooth, wherein
American 556K, American 205K and American Kerry868: soft;
DATEM1#, DATEM-2# and DATEM-4#: moderate.

In conclusion, the products by the present invention can be used as additives in baking food, and the comprehensive evaluation for the baking food with additives of the present invention is superior to or equal to the foreign baking food with other similar additives.

### Baking Test 2

American 556K is used in controlled group. Test processes and results are listed as follows.

### Formula:

| Flour | Dry yeast | Sugar | Salt | Grease | Egg | Milk powder | Water |
|---|---|---|---|---|---|---|---|
| 100 g | 12 g | 18 g | 1 g | 6 g | / | / | 55 g |

Operation conditions:

| Portion | Intermediate | Final fermentation | | | Baking | | |
|---|---|---|---|---|---|---|---|
| weight | fermentation | Temperature | Humidity | Time | Broiling | Baking | Time |
| 115 g | 15 min | 38° C. | 85% | 115 min | 185° C. | 195° C. | 15 min |

1. Quality evaluation (according to GB/14644-93):

| | | Quality grade | | | | | | | | | |
|---|---|---|---|---|---|---|---|---|---|---|---|
| | | | | | Crust | | | | | | |
| | | Volume | | | character & | | | | | | |
| | | Specific | | Crust | Bread | Crumb | | | | | Total |
| Flour & | | votume | Score | color | traits | color | Smoothness | Texture | Elasticity | Taste | score |
| Additives | | cm³/g | (35) | (5) | (5) | (5) | (10) | (25) | (10) | (5) | (100) |
| DATEM-4# | 3000 ppm | 6.25 | 22.5 | 4 | 4 | 3 | 8 | 18 | 8 | 4 | 71.5 |
| DATEM-1# | 3000 ppm | 6.13 | 21.3 | 4 | 4 | 4 | 8 | 19 | 8 | 4 | 72.3 |
| 556K | 3000 ppm | 6.16 | 21.6 | 4 | 4 | 3 | 7 | 17 | 8 | 4 | 67.6 |

### Analysis:

According to the quality evaluation, the comprehensive quality evaluation results for the baking products produced with different additives are as follows: DATEM-1#>DATEM-4#>556K.

2. Toast Volume (Kerry868 is not used in the):

| | |
|---|---|
| American 556K: 610/97.5; | DATEM1#: 610/98.9; |
| DATEM-4#: 610/99.5. | |

3. Bun height (Kerry868 is not used in the):

| | |
|---|---|
| DATEM-4#: 5.58/7.98; | American 556K: 4.88/8.53; |
| DATEM1#: 5.22/8.02. | |

4. Fermentation time: all are 3 hours.

In conclusion, the products by the present invention can be used as additives in baking food, and the comprehensive evaluation for the baking food with additives of the present invention is superior to or equal to the foreign baking food with other similar additives.

### French Bread Test

Danisco556K, Danisco205K, Kerry868 and EMWL322 are used in controlled groups. Operation conditions and results are listed as follows. The formula and operation conditions are the same as the baking test 2.

Quality evaluation:

| | Quality grade | | | | | |
|---|---|---|---|---|---|---|
| | Crust conditions | | | Crumb conditions | | |
| Flour & | Smooth- | Brittleness | Texture | Color | Color | Bubble |
| Additives | ness (5) | (5) | (5) | (5) | (5) | (5) |
| Danisco556K | 4 | 4 | 4 | 4 | 4 | 3 |
| Danisco205K | 4 | 3 | 4 | 4 | 3 | 2 |
| Kerry868 | 4 | 4 | 5 | 3 | 4 | 4 |
| EMWL322 | 4 | 4 | 3 | 4 | 4 | 4 |
| DATEM-1# | 4 | 4 | 5 | 4 | 4 | 5 |
| DATEM-2# | 4 | 4 | 4 | 4 | 4 | 4 |
| DATEM-3# | 4 | 4 | 5 | 4 | 4 | 4 |
| DATEM-4# | 4 | 4 | 4 | 5 | 4 | 4 |

Test results:
1. Drop test (breads are baked after free falling from a height of 12 cm):
The overall effects are as follows: DATEM-1#, DATEM-4#>Kerry868, DATEM-2#, DATEM-3#>Danisco556K, EMWL322>Danisco205K
2. The volume before falling:

| | |
|---|---|
| Danisco556K: 330 cm³; | Danisco205K: 360 cm³; |
| Kerry868: 310 cm³; | EMWL322: 310 cm³; |
| DATEM-1#: 410 cm³; | DATEM-2#: 360 cm³; |
| DATEM-3#: 330 cm³; | DATEM-4#: 410 cm³. |

3. The volume after falling:

| | |
|---|---|
| Danisco556K: 230 cm³; | Danisco205K: 300 cm³; |
| Kerry868: 240 cm³; | EMWL322: 250 cm³; |
| DATEM-1#: 300 cm³; | DATEM-2#: 320 cm³; |
| DATEM-3#: 330 cm³; | DATEM-4#: 370 cm³. |

There are no significant differences of the fermentation resistance and smoothness between each groups.

In conclusion, the products by the present invention can be used as additives in baking food, and the comprehensive evaluation for the baking food with additives of the present invention is superior to or equal to the foreign baking food with other similar additives.

It is to be understood that the present invention includes but is not limited to the disclosed embodiments. The scopes of the appended claims encompass all the modifications and the equivalents which are apparent to those skilled in the art.

1. A method for the preparation of diacetyl tartaric acid esters of mono- and di-glycerides, comprising steps of:
A. adding L-tartaric acid and/or DL-tartaric acid and acetic anhydride into a reaction kettle, adding concentrated phosphoric acid as catalyst and mixing together to form a reaction system;
B. heating the reaction kettle until a temperature of the reaction system reaches 50° C.˜55° C. to initiate a reaction, after the temperature of the reaction system rises and then drops to 80° C.˜90° C., keeping the temperature at 80° C.˜90° C. for 20˜60 min to obtain an intermediate product; and
C. adding stearic acid esters of mono- and di-glycerides to the intermediate product, then adding powdery sodium hydroxide as catalyst, controlling an inner pressure of the reaction kettle at −0.09 Mpa˜−0.098 Mpa, heating the reaction kettle again until a temperature of the reaction system reaches 90° C.˜130° C., and keeping the temperature at 90° C.˜130° C. for 20˜40 min to obtain said diacetyl tartaric acid esters of mono- and di-glycerides.

2. A method according to claim 1, wherein a weight ratio between said acetic anhydride and said L-tartaric acid and/or DL-tartaric acid is 1.5˜3.5:1.

3. A method according to claim 1, wherein said acetic anhydride in step A is added into the reaction system in twice.

4. A method according to claim 1, wherein an amount of said concentrated phosphoric acid accounts for 0.004%˜0.005% of a total amount of said L-tartaric acid and/or DL-tartaric acid, and an amount of said powdery sodium hydroxide accounts for 0.004%˜0.005% of a total amount of said L-tartaric acid and/or DL-tartaric acid.

5. A method according to claim 1, wherein said stearic acid esters of mono- and di-glycerides have a freezing point of 60° C.˜70° C. and an iodine value not more than 3 g/100 g, and a amount of stearic acid ester of monoglyceride accounts for 50%˜90% of a total amount of said stearic acid esters of mono- and di-glycerides.

6. A method according to claim 1, wherein a weight ratio between said stearic acid esters of mono- and di-glycerides and said L-tartaric acid and/or DL-tartaric acid is 2.5˜4:1.

7. A method according to claim 1, wherein said diacetyl tartaric acid esters of mono- and di-glycerides have an acid value of 60˜105 mgKOH/g and a saponification value of 300˜550 mgKOH/g.

8. A method according to claim 1, wherein said step C further includes a process of spray prilling said diacetyl tartaric acid esters of mono- and di-glycerides at the temperature of 90° C.˜110° C. under atmospheric pressure, to make a white solid of said diacetyl tartaric acid esters of mono- and di-glycerides.

9. An application of said diacetyl tartaric acid esters of mono- and di-glycerides produced by a method according to any one of claims 1˜8 as baked food emulsifiers, wherein said diacetyl tartaric acid esters of mono- and di-glycerides are added with anticaking agents.

10. An application according to claim 9, wherein said anticaking agent includes at least one of 90% stearic acid monoglyceride, fluent starch and food grade silicon dioxide and tricalcium phosphate.
